(19) United States
(12) Patent Application Publication
(10) Pub. No.: US 2020/0309706 A1
(43) Pub. Date: Oct. 1, 2020

(54) NANO-OPTICAL PLASMONIC CHIP FOR THE DETECTION OF SUBSTANCES OR MOLECULES IN THE ENVIRONMENT, FOOD, AND BIOLOGICAL SYSTEMS

(71) Applicant: SAFTRA PHOTONICS, S.R.O., Kosice (SK)

(72) Inventors: Santiago SANCHEZ-CORTES, Collado-Villalba, Madrid (ES); Pavol MISKOVSKÝ, Bratislava (SK); Daniel JANCURA, Humenné (SK)

(73) Assignee: SAFTRA PHOTONICS, S.R.O., Kosice (SK)

(21) Appl. No.: 16/772,669

(22) PCT Filed: Dec. 13, 2018
(86) PCT No.: PCT/IB18/60065

§ 371 Date: Jun. 12, 2020

(30) Foreign Application Priority Data
Dec. 14, 2017 (SK) PP 127-2017

(51) Int. Cl. G01N 21/65; G01N 21/27; G01N 33/531

(52) U.S. Cl. CPC G01N 21/658; G01N 33/531; G01N 21/278

(57) ABSTRACT

A portable nano-optical chip based on the principle of generating plasmons for detecting very low concentrations of substances/molecules (down to the single-molecule level) in the environment (water, air, soil), food, and biological systems is disclosed. The nano-optical chip comprises a substrate (1) on which plasmonic nanoparticles (5) are immobilized with a selected distance between the individual nanoparticles, e.g., by pulsed laser deposition, wherein the distance is selected such that hot spots (4) are formed in the gaps between the nanoparticles. Both selectivity and sensitivity of thus created nanoparticle surface for the detection of molecules to be analyzed are modulated and increased by the functionalization process, which consists in binding specific linkers (3), such as cavitand linkers or bifunctional linkers, to the nanoparticles. The use of bifunctional linkers enables deposition of one or more additional layers of plasmonic nanoparticles.

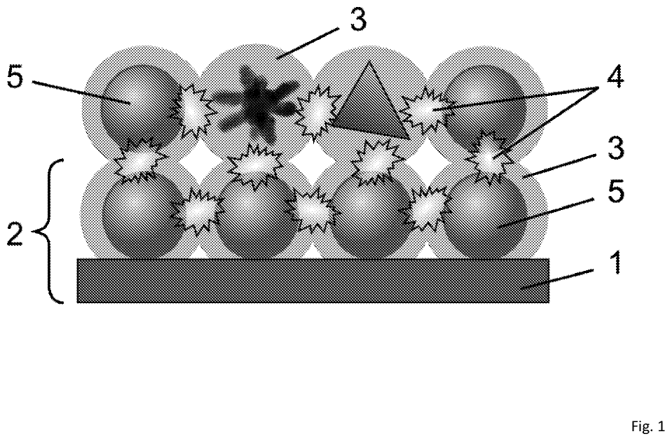

### FIELD OF THE INVENTION

The patent pertains to the structure of a portable nano-optical chip based on the principle of generating plasmons and on the modification of a plasmonic nanoparticle surface. The nano-optical chip detects very low concentrations of substances/molecules in the environment (water, air, soil), food, and biological systems.

### DESCRIPTION OF THE RELATED ART

Plasmons are oscillations of electron plasma that are excited by light on metal nanoparticles; the excitation results in generating a significantly enhanced electromagnetic field (EF) on the surface of the nanoparticles. SERS (Surface-enhanced Raman spectroscopy) is a technique based on significant enhancement of EF on metal nanostructures and subsequent increase in the intensity of Raman signal. Such increased Raman signal transforms Raman spectroscopy from a structural analytical method into a structurally sensitive nano-probe able to detect very low concentration of molecules down to the single-molecule level.

At present, SERS is the only single-molecule detection option with a simultaneous analysis of the chemical structure. Technically, SERS depends on the existence of the so-called “hot spots” (HS) found in the structure of plasma nanoparticles. We recognize two different types of HS: a) gaps between metallic nanoparticles and b) spikes of nanoparticle surface exhibiting a high surface curvature. In both cases, the EF is strongly enhanced by the excitation light. Thus, enhanced EF significantly increases Raman signal from the molecules found in these HS.

### SUMMARY OF THE INVENTION

Description of the Nano-Optical Chip:

Plasmonic nanoparticle surface created by physical methods, such as pulsed laser deposition, functionalized by specific molecular linkers and by the deposition of additional layer/layers of nanoparticles of various shapes.
1. Plasmonic nanoparticle surface: The plasmonic nanoparticle surface of the developed chip is composed of plasmonic nanoparticles (NPs) deposited on a substrate, which is achieved by physical methods, for example, by the method of pulsed laser deposition (PLD). Such methods ensure the homogeneous distribution of the NPs as well as the selected distance between individual NPs on the substrate; e.g. when using the PLD method, this is achieved by means of regulating the power and frequency of the laser, which determines the number of HS created and, consequently, also the sensitivity of the chip.
2. Functionalization of the plasmonic nanoparticle surface: The functionalization of the plasmonic nanoparticle surface by molecular linkers increases the surface affinity for the molecules to be detected. Such functionalization is carried out by the following linkers: a) cavitand linkers (CL) capable of binding specific molecules by means of an inclusion mechanism caused by the existence of internal cavities in these molecules. The functionalization of plasmonic nanoparticle surfaces by these cavitands requires the use of specific molecular groups to ensure their interaction with the plasmonic nanoparticle surface; b) bifunctional linkers (BL). The bifunctional linkers are used for connecting the NPs with suitable distances or gaps between the NPs, which leads to the creation of HS in the gap between the individual nanoparticles. These molecular linkers also provide a suitable environment for the binding of a large number of hydrophobic molecules to be detected. The use of bifunctional molecules also enables creating the second and additional layers of NPs, which leads to the formation of additional HS between the layers of NPs, c) by other molecules generating favorable conditions for selective binding of the molecules to be detected.

### OVERVIEW OF THE DRAWINGS

FIGURE: Schematic representation of the structure of the nano-optical chip

### DETAILED DESCRIPTION OF THE INVENTION

The nano-optical chip integrates two different parts: the plasmonic nanoparticle surface consisting of plasmonic nanoparticles deposited on the substrate and the molecular functionalization of the plasmonic nanoparticle surface.

The plasmonic nanoparticle surface 2 comprises suitably shaped and spaced plasmonic nanoparticles 5 (NPs 5) immobilized on the substrate 1. Depending on the type of NPs 5 deposited on the substrate 1 and the spacing between them, an optimal amount of HS 4 is generated, where the EF is strongly enhanced by the interaction between the light and plasmons.

Both selectivity and sensitivity of thus created plasmonic nanoparticle surface 2 for the detection of substances/molecules are increased by the molecular functionalization 3 of the plasmonic nanoparticle surface 2. The most suitable functionalization is achieved using the following linkers: i) cavitand linkers (CL) containing internal cavities in their structure. CL molecules are bound directly to the surface and they lead to highly specific recognition and binding of the molecules to be detected; ii) bifunctional linkers (BL) containing aliphatic chains or other molecules creating favorable conditions for the selective binding of the molecules to be detected.

The subsequent increase in the sensitivity and selectivity of the nano-optical chip lies in the possibility of attaching a second layer of NPs 5 with different morphology (shape), such as round NPs, pyramidal NPs, star-like NPs to the primary functionalized plasmonic nanoparticle surface 2. The aim is to increase the size of the surface available for binding the substances/molecules to be detected while increasing the number of HS in the nano-optical chip. In addition, the functionalization of the second layer of NPs 5 creates favorable conditions for the binding of other molecules to be detected.

### INDUSTRIAL USABILITY

Nano-optical chips can detect the substances/molecules in the environment (water, air, soil), food, and biological systems. The detection and identification of these substances/molecules by certified techniques (mass spectrometry or gas chromatography) is time-consuming and expensive. In comparison with the certified methods (mass spectrometry or gas chromatography), the detection of substances/molecules by nano-optical chips is cheaper, faster, more sensitive and performed on the spot (without the need for pre-treatment of samples in the laboratory).

### List of Abbreviations

EF Electromagnetic field
SERS Surface-enhanced Raman spectroscopy
HS Hot spots, (areas of high-intensity of EF)
PLD Pulsed laser deposition
NPs Plasmonic nanoparticles
BL Bifunctional linkers
CL Cavitand linkers

1. The structure of the nano-optical chip for the detection of substances/molecules in the environment, food, and biological systems is characterized by being composed of a plasmonic nanoparticle surface 2 that consists of plasmonic nanoparticles 5 deposited on the substrate 1 with a selected distance between the individual nanoparticles.

2. The structure of the nano-optical chip for the detection of substances/molecules in the environment, food, and biological systems according to claim 1 is characterized in that the primary plasmonic nanoparticle surface 2 is functionalized 3 by specific CL, BL linkers and/or other molecules that create favorable conditions for binding and detection of selected molecules on the primary plasmonic nanoparticle surface.

3. The structure of the nano-optical chip for the detection of substances/molecules in the environment, food, and biological systems according to claims 1 and 2 is characterized by the deposition of additional layer/layers of plasmonic nanoparticles 5 with a selected shape of individual nanoparticles.

4. The structure of the nano-optical chip for the detection of substances/molecules in the environment, food, and biological systems according to claims 1, 2, and 3 is characterized by the functionalization 3 of the second/additional layers/layers of plasmonic nanoparticles 5 by specific CL, BL linkers, and/or other molecules that create favorable conditions for binding and subsequent detection of substances/molecules on the secondary and/or additional plasmonic nanoparticle surface/surfaces.
